(19) United States
(12) Patent Application Publication
(10) Pub. No.: US 2021/0106711 A1
(43) Pub. Date: Apr. 15, 2021

(54) IRON OXIDE NANOWIRES BASED FILTER FOR THE INACTIVATION OF PATHOGENS

(71) Applicant: VIRGINIA COMMONWEALTH UNIVERSITY INTELLECTUAL PROPERTY FOUNDATION, Richmond, VA (US)

(72) Inventors: Weining Wang, Glen Allen, VA (US); Dawei Wang, Richmond, VA (US); Ping Xu, Henrico, VA (US); Bin Zhu, Richmond, VA (US)

(21) Appl. No.: 17/047,536

(22) PCT Filed: Apr. 18, 2019
(86) PCT No.: PCT/US19/28063

§ 371 Date: Oct. 14, 2020

Related U.S. Application Data
(60) Provisional application No. 62/659,335, filed on Apr. 18, 2018.

(51) Int. Cl. A61L 9/014; B01J 20/02; B01J 20/06; B01J 20/28; B01J 20/30; A61L 9/16; F24F 3/16

(52) U.S. Cl. CPC A61L 9/014; B01J 20/0229; B01J 20/06; B01J 20/28038; B01J 20/28004; A61L 2209/22; B01J 20/3071; B01J 20/3078; A61L 9/16; F24F 3/16; A61L 2209/14; B01J 20/28052

(57) ABSTRACT

Disclosed herein are embodiments of filtration systems and iron oxide nanowire-based filter meshes that can capture and inactivate pathogens in air. The filter meshes can include a porous lattice of iron metal and iron oxide nanowires radiating from the porous lattice of iron metal. The iron oxide nanowires radiating from the porous lattice of iron metal can be created by processing the filter mesh using the disclosed method. Pathogens can be inactivated by passing a sample containing the pathogens through the filter mesh and inactivating at least a portion of the pathogens as the sample passes through the filter mesh.

### RELATED APPLICATIONS

This application claims benefit of U.S. Provisional Application No. 62/659,335, filed Apr. 18, 2018, which is hereby incorporated herein by reference in its entirety.

### STATEMENT REGARDING FEDERALLY SPONSORED RESEARCH OR DEVELOPMENT

This invention was made with Government Support under Grant No. R01DE023078 awarded by the National Institutes of Health. The Government has certain rights in the invention.

### FIELD

The present disclosure relates to the field of filters. Furthermore, the present disclosure relates to capturing and inactivating pathogens as they pass through air filters.

### BACKGROUND

The increased amount of time spent indoors by people (from 80% to 90% over the last years in the U.S.) have resulted in a strong demand for improved indoor air quality. However, both the human activities and wide use of chemicals in built environment produce particulate and gaseous pollutants in indoor air, which causes serious health problems. One of the most common methods to improve indoor air quality is to increase the ventilation rates through heating, ventilation, and air conditioning (HVAC) systems. Increased ventilation benefits not only the dilution of indoor air pollutants but also the control of particulate matters with the aid of HVAC filters. However, HVAC systems can also become a microbial breeding ground. For example, under relatively wet (>80% relative humidity (R.H.)) and warm (>12° C.) outdoor air conditions, a proliferation of bacteria on the filter occurred with a subsequent release into the filtered air. In consequence, increasing attention has been paid to prohibit the growth of bacteria and other pathogens in HVAC systems and the subsequent release of bacteria into indoor environment.

Ultraviolet (UV) irradiation is one of the promising methods due to its high efficiency in bioaerosol control. The high energy of UV light results in the damage of the RNA/DNA of bacteria. However, the installation of UV lights should be very careful to avoid any potential risks to occupants, thus limiting its applications. Several other emerging technologies have also been proposed, such as photocatalytic oxidation, plasma, and microwave. Specifically, photocatalytic oxidation produces reactive oxygen species (ROS), such as hydroxyl radicals (.OH), to disinfect bioaerosols. However, this technology needs a complete renovation of the current HVAC system to make light available. The plasma and microwave methods generally require high voltage/power and are thus energy inefficient. In addition, all the above efforts require either a new functional unit (UV, photocatalysis, and microwave), or the transformation of current HVAC systems (plasma). Thus, a safer and more cost-efficient approach to prohibit the growth of bacteria and other pathogens in HVAC systems is needed.

### SUMMARY

The use of filtration systems that include iron oxide nanowires-based filters can address many of the problems discussed above. Methods of making and using these systems are disclosed herein. The details of one or more embodiments of the invention are set forth in the accompanying drawings and the description below. Other features, objects, and advantages of the invention will be apparent from the description and drawings, and from the claims.

Filter meshes including a porous lattice of iron metal and iron oxide nanowires radiating from the porous lattice of iron metal are disclosed herein. In some embodiments, the nanowires have a diameter of no more than 300 nanometers. In some embodiments, the nanowires have length of at least 3 micrometers. The porous lattice can include reactive oxygen species.

Filtration systems, for example, air filtration systems, utilizing the disclosed filter meshes are also disclosed herein. The filtration systems include a housing having inlet and outlet. At least one filter mesh is disposed between the inlet and the outlet. In some embodiments, a plurality of filter meshes are arranged in sequence between the inlet and the outlet. In some embodiments, at least three filter meshes are arranged in sequence. In some embodiments, the filter mesh is in electrical communication with a power supply that is configured to apply a voltage to the filter mesh.

Methods for the inactivation of pathogens in a sample, for example, an air sample, are also disclosed herein. The methods include: providing a filter mesh comprising a porous lattice of iron metal and iron oxide nanowires radiating from the porous lattice of iron metal; passing the sample containing pathogens through the filter mesh; and inactivating at least a portion of the pathogens as the sample passes through the filter mesh. In some embodiments, inactivating at least a portion of the pathogens includes lysing pathogen cell membranes. Passing the sample through the filter mesh can include passing the sample through a plurality of filter meshes arranged in sequence.

In some embodiments, the method for the inactivation of bacteria can include applying a voltage to the filter mesh. The voltage can be, for example, at least 0.1 Volts. In some embodiments, the method can include heating the filter mesh.

In some embodiments, the pathogen is a Gram-positive bacteria. In some embodiments, the pathogen is a Gram-negative bacteria.

Methods of manufacturing the filter meshes are also disclosed herein. The methods can include: providing a porous lattice of iron metal; washing the porous lattice of iron metal with hydrochloric acid; rinsing the porous lattice of iron metal with water; drying the porous lattice of iron metal; and heating the porous lattice of iron metal to a temperature ranging from 600° C. to 900° C.

In some embodiments, the hydrochloric acid is at least 0.1 M hydrochloric acid. In some embodiments, the drying is performed with a vacuum desiccator. In some embodiments, the porous lattice of iron metal is heated for a time period of from 5 hours to 7 hours. In some embodiments, the heating occurs at a rate wherein the temperature rises about by about 3° C./minute to about 10° C./minute.

### DESCRIPTION OF DRAWINGS

The device is explained in even greater detail in the following drawings. The drawings are merely exemplary to illustrate the structure of garments and certain features that may be used singularly or in combination with other features. The drawings are not necessarily drawn to scale.

FIG. 1A shows a cross-sectional, perspective view of a filtration system embodiment including a filter mesh comprising iron nanowires. FIG. 1B shows a schematic illustration of the experimental set-up for the generation and inactivation of S. epidermidis bioaerosols. The applicability of resuspending the filter into PBS buffer to measure the bacteria concentration was verified by a controlled experiment. Firstly, the bacteria amount in the exhaust PBS buffer (N_{buffer-1}) was measured when no IO nanowires (NWs) filter was employed, the operation time was set to be 30 s. Then, the bacteria concentration in the exhaust PBS buffer (N_{buffer-2}) and that on the IO NWs filter (N_{filter}, by resuspending the filter into PBS) was measured when one filter was placed in the front of exhaust buffer. The operation time was also 30 s (no voltage was applied). It was found that N_{buffer-1}≈N_{buffer-2}+N_{filter}. As a result, the above measurement method for estimating the bacteria amount on the filter by resuspending into PBS was applicable. Meanwhile, the IO NWs filter is proven to be of no disinfection ability when no voltage is applied in this way.

FIGS. 2A-2F show some results of the treatment to form IO NWs. Digital images of the pristine iron mesh (FIG. 2A) and iron mesh with IO NWs (FIG. 2B). Optical microscopy image (FIG. 2C), SEM image (FIG. 2D), and TEM image (FIG. 2E) of the IO NWs on the iron mesh. (FIG. 2F) XRD patterns. Scale bars in (FIG. 2C), (FIG. 2D), and (FIG. 2E) represent 200 μm, 5 μm, and 200 nm, respectively.

FIGS. 3A and 3B show optical microscopy images of pristine iron mesh under (FIG. 3A) low and (FIG. 3B) high magnification. Scale bars represent 200 μm.

FIG. 4 shows bacteria concentrations measured by resuspending the filter into the PBS solution. The corresponded bacteria amount can be obtained by multiplying the concentration by the volume of PBS solution (20 mL).

FIGS. 5A-5F show results of inactivation efficiency assays. (FIG. 5A) Inactivation efficiency of IO NWs filter under different conditions. (FIG. 5B) Control experiments using pristine iron mesh, operation time was 10 s. Fluorescence microscope images of S. epidermidis before treatment (control) (FIG. 5C) and after treatment (4.5 V, 10 s) (FIG. 5D). (FIG. 5E) and (FIG. 5F) are the flow cytometry results of samples in (FIG. 5C) and (FIG. 5F). The scale bars in (FIG. 5C) and (FIG. 5D) represent 20 μm.

FIGS. 6A-6D show photographs of bacterial cells before and after treatment. SEM images of S. epidermidis cells before (FIG. 6A) and after (FIG. 6B) treatment. TEM images of S. epidermidis cells before (FIG. 6C) and after (FIG. 6D) treatment. Scale bars in (FIG. 6A), (FIG. 6B), (FIG. 6C), and (FIG. 6D) represent 1 μm. Scale bars in the insets represent 500 nm.

FIG. 7 shows FTIR spectra of bacteria before treatment (black curve) and after treatment (red curve).

FIGS. 8A-8D show results of assays testing various inactivation mechanisms of action. (FIG. 8A) Evolution of fluorescence spectra for the detection of .OH with time. (FIG. 8B) Effect of R.H. on the log inactivation efficiency of S. epidermidis by IO NWs filter, the voltage was 4.5 V and the treatment time was 10 s. (FIG. 8C) Simulated temperature distribution around the filter, air flow rate=0.005 m/s, unit in the scale bar is ° C. (FIG. 8D) Simulated electrical field near an IO NW, the voltage was set to be 4.5 V. Scale bars in (FIG. 8C) and (FIG. 8D) represent 600 μm and 5 μm, respectively.

FIGS. 9A and 9B are photographs showing the effect of DMSO on the bacteria. (FIG. 9A) is the fresh bacteria, (FIG. 9B) is the bacteria mixed with PBS solution of DMSO (100 mM) for 5 min. No significant difference between the two samples was observed, indicating that DMSO is not lethal to the bacteria.

FIG. 10 shows the effect of DMSO on the inactivation performance. Under voltages of 1.5 V and 3.0 V, only 100 mM of DMSO was used because this amount is enough to quench ROS as verified at 4.5 V.

FIG. 11 shows the bulk surface temperatures of the IO NWs filter varied with different applied voltages (air flow velocity=0 m/s).

FIG. 12 shows a three-view drawing of the simulation unit for temperature gradient.

FIGS. 13A and 13B show the effect of air flow rate on the temperature gradient near the IO NWs filter. (FIG. 13A) flow rate=0.5 m/s. and (FIG. 13B) flow rate=5 m/s.

FIGS. 14A and 14B show IO nanoparticles on iron mesh. (FIG. 14A) SEM image and (FIG. 14B) XRD pattern. IO nanoparticles on iron mesh were obtained by heating the mesh in the air to 700° C. from room temperature (5° C./min). Once the temperature reached 700° C. the mesh was taken out from the furnace.

FIG. 15 is a schematic illustration of the inactivation mechanism of S. epidermidis.

FIGS. 16A-16D. (FIG. 16A) The effect of filter number on the capture ratio. (FIG. 16B) Recycle performance of single IO NWs filter. (FIG. 16C) Digital image of the samples before (left, condition: 0 V, 30 min) and after treatment (right, condition: 4.5 V, 30 min) stained by crystal violet. (FIG. 16D) Corresponding bacterial concentration of (FIG. 16C) measured by a hemocytometer.

FIG. 17. (FIG. 17A) XRD pattern. (FIG. 17B) XPS spectra for Fe 2p, (FIG. 17C) SEM image, and (FIG. 17D) TEM image of the IO NWs after five cycles of 1 h operation. Scale bars in (FIG. 17C) and (FIG. 17D) represent 5 μm and 500 nm, respectively.

### DETAILED DESCRIPTION

Terms used throughout this application are to be construed with ordinary and typical meaning to those of ordinary skill in the art. However. Applicant desires that the following terms be given the particular definition as defined below.

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and claims, the singular form “a,” “an,” and “the” include plural references unless the context clearly dictates otherwise. For example, the term “a cell” includes a plurality of cells, including mixtures thereof.

As used in the specification and claims, the singular form “a,” “an,” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the term “nanowire” generally refers to any elongated conductive or semiconductive material (or other material described herein) having an aspect ratio (length:width) of about 10 or more (for example, an aspect ratio of about 10 or more, of about 50 or more, of about 100 or more and of about 1000 or more).

As used herein, an “aspect ratio” is the length of a first axis of a nanostructure divided by the average of the lengths of the second and third axes of the nanostructure, where the second and third axes are the two axes whose lengths are most nearly equal to each other. For example, the aspect ratio for a perfect rod would be the length of its long axis divided by the diameter of a cross-section perpendicular to (normal to) the long axis.

Generally speaking, a nanostructure indicates that the diameter of the structure is in the order of nanometers, typically around several hundred nanometers or less. It should be appreciated that although nanowires are frequently referred to, the techniques described herein are also applicable to other nanostructures, such as nanorods, nanotubes, nanotetrapods, nanoribbons and/or combinations thereof.

While the disclosed devices and methods are described in the context of bacterial inactivation, it is understood that this is meant to be exemplary only. Similar inventive principles and concepts can apply to the inactivation of other pathogens. As used herein, the term “pathogen” can refer to any organism of microscopic or ultramicroscopic size including, but not limited to, bacteria, viruses, fungi and protozoa.

As used herein. “inactivation” of a pathogen can mean killing a pathogen or rendering the pathogen partially or completely immobilized (i.e., capturing pathogens).

Heating, ventilation, and air conditioning (HVAC) systems are among the most common methods to improve indoor air quality. However, after long-term operation, the HVAC filter can result in a proliferation of bacteria, which release into the filtered air subsequently. As mentioned above, several technologies have been proposed to prohibit the growth of bacteria, including UV irradiation, photocatalytic oxidation, plasma, and microwave. However, these technologies require a complete renovation of the current HVAC system. A more feasible approach is modification of the existing air filters. Incorporating an antimicrobial layer achieves efficient control of indoor air bioaerosol (Yale et al., 1968). A few studies have reported the modification of air filters for antimicrobial properties, such as decorating or coating of chemicals or Ag-based nanomaterials onto the filter (Miaskiewicz-Peska et al., 2011; Ko et al., 2014). The stability of the filters modified by these processes is an inherent issue. These bonds between the chemicals and nanomaterials are often loose and weak. Under long term use or high air flow rate, these added materials may detach, causing secondary contaminations that cause potential risks to human health. Furthermore, these chemicals/materials are relatively expensive. Therefore, a safe and cost-efficient approach for improving the antimicrobial properties of an air filter is needed.

Disclosed herein is a filtration system 10 and an iron oxide nanowire-based filter mesh 2 and that can capture and inactivate pathogens in air. The filter mesh comprises a porous lattice of iron metal and iron oxide nanowires radiating from the porous lattice of iron metal. The iron oxide nanowires-based filter meshes 2 are stable under normal environmental conditions and operations. Furthermore, the costs of iron metal and the manufacturing process of growing the of iron oxide nanowires costs less than the Ag-based filters described above.

The iron oxide nanowires radiating from the porous lattice of iron metal are created by processing an iron metal filter mesh using the methods disclosed herein. The in-situ growth of nanowires directly out of the iron mesh greatly enhances the long-term stability of the filter mesh as compared to the more conventional processes of coating and decorating filter meshes with antimicrobial agents.

Pathogens in a sample can be inactivated by passing the sample containing pathogens through the filter mesh and inactivating at least a portion of the pathogens as the sample passes through the filter mesh. The long aspect ratio of the nanowires enhances the active surface area and induces strong electric current and heat transfer rate, which contribute to the efficient inactivation of airborne pathogens.

While described in the context of HVAC systems, it will be understood that the concepts and ideas disclosed herein can be applied to many applications where it is beneficial to inactivate airborne pathogens. Other examples can include, but are not limited to, healthcare related respiratory devices or masks, free-standing air filtration devices, and robotic mops.

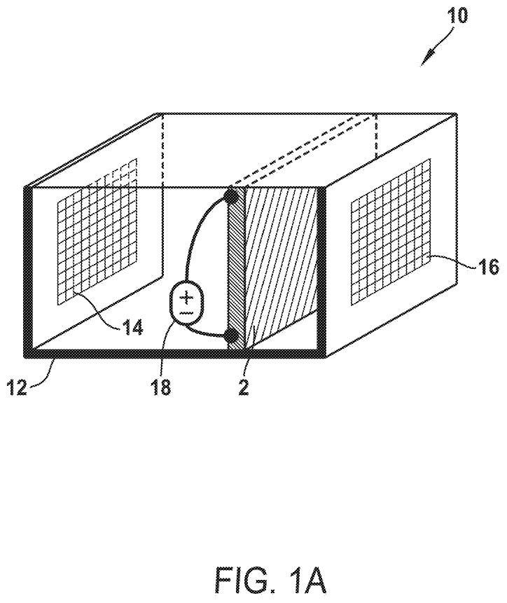

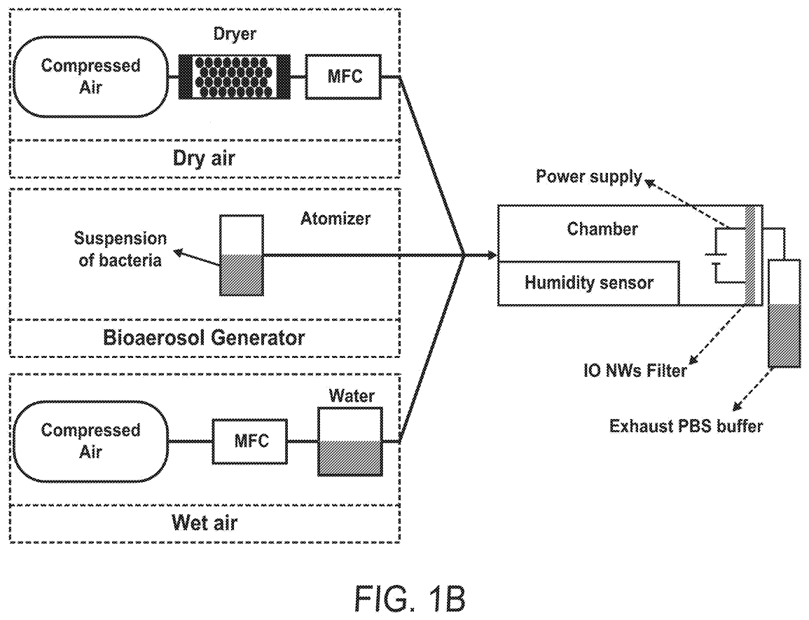

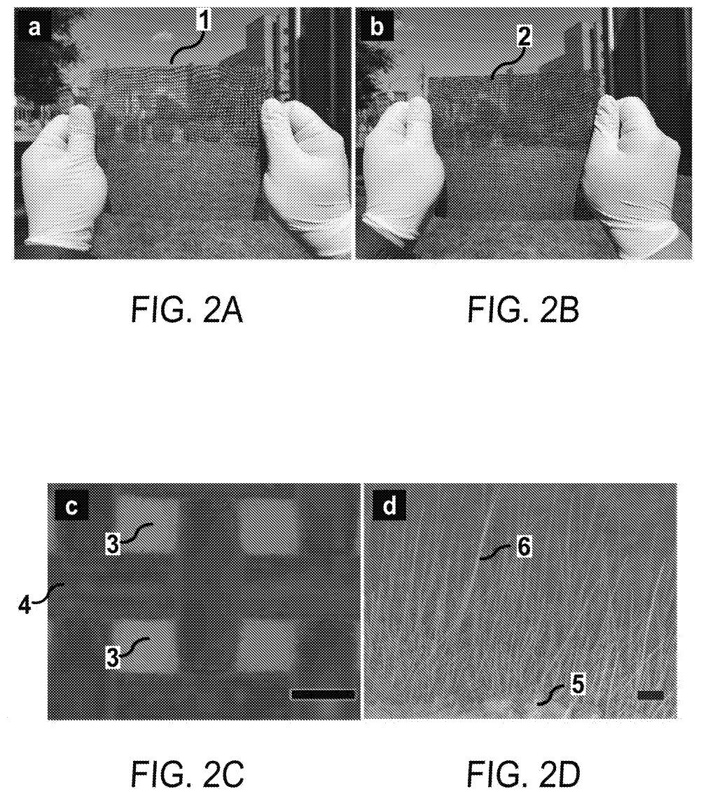

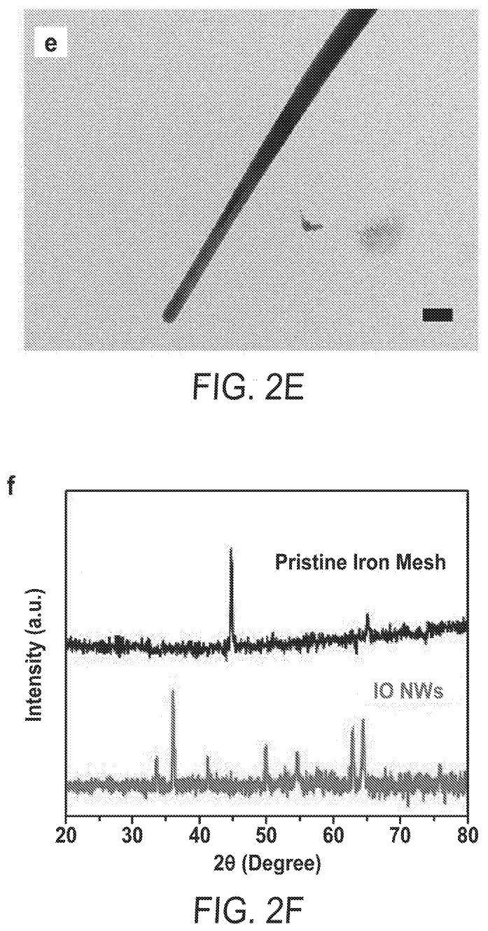

As shown in FIG. 1A, the filtration systems 10 disclosed herein include a housing 12 having an inlet 14 and an outlet 16. At least one filter mesh 2 comprising iron oxide nanowires is positioned between the inlet 14 and the outlet 16. The filter mesh 2 is formed of a porous lattice of iron metal and iron oxide nanowires radiating from the porous lattice of iron metal (described below in reference to FIG. 2). In some embodiments, the filtration system 10 can include a power supply 18 in electrical communication with and configured to apply a voltage to the filter mesh 2. The power supply 18 is shown inside the housing 10 for illustration purposes, but can also be located outside the housing 10. The application of a voltage across filter mesh 2 can induce Joule heating around the filter mesh 2. Together, Joule heating of the local environment coupled with electroporation of passing cells can work together (and in combination with other mechanisms) to inactivate nearby pathogens.

The filter mesh embodiments disclosed herein include a porous lattice of iron metal and iron oxide nanowires radiating from the porous lattice of iron metal. FIG. 2A shows a digital image of a pristine, unmodified iron mesh 1. After thermal treatment using the disclosed method, the color of the modified iron mesh 2 turns burgundy, as depicted in the digital image of FIG. 2B. FIG. 2C shows the optical microscopy image of the modified iron mesh after the thermal treatment. The iron mesh is a web-like construction comprising porous lattice that is made by interlacing iron wires 4 to define filter pores 3. The filter pore size affects the air passing rate and hence the induced back pressure. Filter pores can be, for example, from about 0.01 inch to about 0.9 inches, or from a 1× mesh to a 60×60 mesh. After the thermal treatment, the surface 5 of the modified iron wires 4 is fully covered by nanowires 6, as depicted in the SEM photograph of FIG. 2D. The modification of the filter mesh 2 with iron oxide nanowires 6 can introduce reactive oxygen species, such as, for example, hydroxyl radicals, that can work in combination with other mechanisms to inactivate nearby pathogens.

In some embodiments, the nanowires have a diameter that can range from about 50 nanometers to about 300 nanometers. For example, in some embodiments, the nanowires can have a diameter of about 50 nanometers, of about 75 nanometers, of about 100 nanometers, of about 125 nanometers, of about 150 nanometers, of about 175 nanometers, of about 200 nanometers, of about 225 nanometers, of about 250 nanometers, of about 275 nanometers, and of about 300 nanometers.

In some embodiments, the nanowires have a length of from about 3 micrometers to about 50 micrometers (including, for example, a length of about 3 micrometers, a length of about 6 micrometers, a length of about 9 micrometers, a length of about 12 micrometers, a length of about 13 micrometers, a length of about 15 micrometers, a length of about 18 micrometers, a length of about 21 micrometers, a length of about 24 micrometers, a length of about 27 micrometers, a length of about 30 micrometers, a length of about 35 micrometers, a length of about 40 micrometers, a length of about 45 micrometers, and a length of about 50 micrometers). High-aspect ratio nanowire structures advantageously create electric fields that may be more effective at lysing cells than, for example, shorter aspect ratio nanoparticles.

In some embodiments, the filtration system 10 comprises a plurality of filter meshes 2 arranged in sequence between the inlet 14 and the outlet 16, such that the incoming air is routed through each of the filter meshes 2. In some embodiments, the filtration system 10 comprises at least three filter meshes arranged in sequence (including, for example, at least three filter meshes, at least four filter meshes, at least five filter meshes, at least six filter meshes, at least seven filter meshes, at least eight filter meshes, at least nine filter meshes, and at least ten filter meshes arranged in sequence). As described in the examples, passing the air through a plurality of filter meshes 2 in sequence can increase the pathogen inactivation efficiency of the filtration system 10.

Methods of inactivating pathogens are also disclosed herein. The methods can include: providing an filter mesh comprising a porous lattice of iron metal and iron oxide nanowires radiating from the porous lattice of iron metal; passing a sample (for example, an air sample) containing pathogens through the filter mesh; and inactivating at least a portion of the pathogens as the sample passes through the filter mesh.

As discussed above, applying a voltage across the filter mesh 2 can facilitate the inactivation of pathogens. The voltage can range from about 0.1 Volts to about 50 Volts, including at least about 0.1 Volts, at least about 1.5 Volts, at least about 3 Volts, at least about 4.5 Volts, at least about 5 Volts, at least about 7.5 Volts, at least about 10 Volts, at least about 15 Volts, at least about 20 Volts, at least about 25 Volts, at least about 30 Volts, at least about 35 Volts, at least about 40 Volts, at least about 45 Volts, and at least about 50 Volts.

In some embodiments, the method can capture or inactivate at least 90% (e.g., at least 90%, at least 91%, at least 92%, at least 93%, at least 93%, at least 94%, at least 95%, at least 96%, at least 97%, at least 98%, or at least 99%) of the pathogens as the sample passes through the filter mesh.

In some embodiments, the pathogen is a Gram-positive bacteria. In some embodiments, the pathogen is a Gram-negative bacteria. In some embodiments, the bacteria is Escherichia coli, M. tuberculosis, M. bovis, M. avium, M. intracellulare, M. africanum, M. kansasii. M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium dficile, other Clostridium species, Yersinia enterolitica, and other Yersinia species. In some embodiments, the bacteria comprises Staphylococcus epidermidis. In some embodiments, the bacterial comprises Escherichia coli. The inactivated pathogens can be any mixture of different types of bacteria, viruses, fungi, and protozoa.

Methods of manufacturing the filter meshes comprising iron oxide nanowires are also disclosed herein. The methods of manufacturing include providing a porous lattice of iron metal; washing the porous lattice of iron metal with hydrochloric acid; rinsing the porous lattice of iron metal with water; drying the porous lattice of iron metal; and heating the porous lattice of iron metal to a temperature ranging from about 600° C. to about 900° C.

In some embodiments, the hydrochloric acid concentration can range from 0.1 M to 1 M, including, for example, about 0.1 M, about 0.2 M, about 0.3 M, about 0.4 M, about 0.5 M, about 0.6 M, about 0.7M, about 0.8 M, about 0.9 M, and about 1 M hydrochloric acid. In some embodiments, the hydrochloric acid removes an oxide layer of the porous lattice of iron metal. In some embodiments, the drying is performed with a vacuum desiccator.

In some embodiments, the porous lattice of iron metal is heated to a high temperature. The temperature can be about 700° C., or can range from about 600° C. to about 900° C. (e.g., about 600° C., about 610° C., about 620° C. about 630° C., about 640° C., about 650° C., about 660° C., about 670° C., about 680° C., about 690° C., about 700° C., about 710° C., about 720° C., about 730° C., about 740° C., about 750° C. about 760° C., about 770° C., about 780° C., about 790° C., about 800° C., about 810° C., about 820° C., about 830° C., about 840° C., about 850° C. about 860° C., about 870° C., about 880° C. about 890° C., or about 900° C.). The porous lattice of iron metal may be heated to the temperature for a time period that can range from about 5 to about 7 hours (e.g., about 5 hours, about 5 hours and 10 minutes, about 5 hours and 20 minutes, about 5 hours and 30 minutes, about 5 hours and 40 minutes, about 5 hours and 50 minutes, about 6 hours, about 6 hours and 10 minutes, about 6 hours and 20 minutes, about 6 hours and 30 minutes, about 6 hours and 40 minutes, about 6 hours and 50 minutes, or about 7 hours).

In some embodiments, the heating occurs at a rate wherein the temperature rises by about 3° C./minute to about 10° C./minute (e.g., about 3° C./minute, about 3.5° C./minute, about 3.6° C./minute, about 3.7° C./minute, about 3.8° C./minute, about 3.9° C./minute, about 4° C./minute, about 4.1° C./minute, about 4.2° C./minute, about 4.3° C./minute, about 4.4° C./minute, about 4.5° C./minute, about 4.6° C./minute, about 4.7° C./minute, about 4.8° C./minute, about 4.9° C./minute, about 5.0° C./minute, about 5.1° C./minute, about 5.2° C./minute, about 5.3° C./minute, about 5.3° C./minute, about 5.4° C./minute, 5.5° C./minute, about 5.6° C./minute, about 5.7° C./minute, about 5.8° C./minute, about 5.9° C./minute, about 6° C./minute, about 6.1° C./minute, about 6.2° C./minute, about 6.3° C./minute, about 6.4° C./minute, about 6.5° C./minute, 6.6° C./minute, about 6.7° C./minute, about 6.8° C./minute, about 6.9° C./minute, about 7° C./minute, about 8° C./minute, about 9° C./minute, or about 10° C./minute).

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1: Materials and Methods

Iron Oxide Nanowires on Iron Mesh. IO nanowires were synthesized on the basis of a recent protocol with modification (Fu et al., 2001). Iron mesh (from McMastcr-Carr, 60×60 mesh, wire diameter=190 μm) was casted into a circular shape with a diameter of 5 cm. The casted iron mesh was then washed with 1 M hydrochloric acid to remove the oxide layer and then rinsed with ultrapure water thoroughly (18.2 MΩ·cm). After drying in a vacuum desiccator, the iron mesh was heated in air at 700° C. for 6 h to grow IO nanowires on the mesh. The temperature rising rate was set to be 5° C./min. For experimental comparisons, IO nanoparticles on iron mesh were obtained by heating the mesh in the air to 700° C. from room temperature (5° C./min). Once the temperature reached 700° C., the mesh was taken out from the furnace.

Inactivation of Bacteria. S. epidermidis (ATTC #14990) was selected because it is found in various built environment and is recommended by ISO 14698-1 for testing the biological efficiency of air samplers. The suspension of S. epidermidis for bioaerosol generation was prepared according to a previous protocol (Park et al., 2013). The nutrient medium was prepared by mixing 5 g of peptone (from Sigma Aldrich), 3 g of meat extract (from Sigma Aldrich), and 1000 mL of ultrapure water. E. coli (ATCC #15597) was grown in Luria-Bertani broth (LB broth: Fisher). E. coli suspension was prepared according to a previous study (Huo et al., 2016).

The set-up of the bacterial inactivation experiment consists of several components, including a bioaerosol generator, a humidity control system, and an inactivation chamber, as schematically shown in FIG. 1B. All the equipment was rinsed by ethanol (70%) and sterilized by UV light irradiation for 10 min before each experiment. In a typical experiment, bioaerosols containing bacteria were generated by an atomizer. Then, bioaerosols were fed into a cylindrical chamber (length=30 cm, diameter=5 cm) with air as the carrier gas. The relative humidity in the chamber was controlled by tuning the flow rate ratio between dry air and wet air. Meanwhile, the total air flow rate was maintained constant (0.5 L/min), ensuring consistent resident time of bacteria in the chamber. The air flow velocity in the chamber was calculated to be ˜0.005 m/s. The R.H. was monitored by a humidity sensor (McMaster, 32705K11). The voltage (0-4.5 V) applied on a single piece of filter was tuned by a home-made DC power supply. In a typical experiment, after running the system for specific times (0-30 s, unless mentioned elsewhere), both the atomizer and power supply were turned off immediately. The IO NWs filter was transferred into 20 mL of phosphate-buffered solution (PBS, 0.1 M) to measure the bacterial concentrations of S. epidermidis on the IO filter (captured). More experimental details are shown in FIG. 1B. The number of bacteria in the exhaust (escaped) was also obtained by measuring its concentration in the exhaust PBS buffer mL, behind the chamber). After being vortexed for 1 min (5000 rpm), each sample was serially diluted, plated in three duplicates, and incubated at 37° C. for 24 h for measurements. Resuspending the filter into the buffer solution to measure the bacteria concentration was verified to be applicable (FIG. 1B).

Characterization and Measurements. The morphology and size of the samples were analyzed with a Hitachi Su-70 field emission scanning electron microscope (FE-SEM). The structure of the samples was analyzed by a JEOL JEM-1230 transmission electron microscope (TEM). The accelerating voltage was set to be 100 kV. To prepare the samples for TEM characterization, IO NWs were scratched from the mesh and then dispersed in ethanol. The ethanol solution was drop casted on a Cu grid for TEM characterization. To prepare the bacterial samples for SEM and TEM analysis, a protocol from a previous study was followed (Huo et al., 2016; W. Wang et al., 2013). Optical images were obtained with an optical microscope (Scope.A1, Zeiss). The crystallinity was characterized by a PANalytical X'Pert Pro MPD X-ray diffractoineter (XRD) equipped with a Cu-Kα radiation source (λ=1.5401 Å). X-ray photoelectron spectroscopy (XPS, Thermo Fisher ESCAab 250) was used to determine the valance state of Fe on the filter. The characterization of surface chemistry of S. epidermidis before and after inactivation was carried out by using a Fourier transform infrared (FTIR) spectrometer (Nicolet iS50, Thermo Fisher Scientific). S. epidermidis was collected from its suspension by centrifugation, dried at 37° C. for 2 h in an oven prior to the FTIR analysis. The strength of fluorescence signal was quantified by a Guava® EasyCyte Flow Cytometer. For fluorescent microscope assay, 1 mL of cells suspensions were centrifuged and resuspended in 10 μl of PBS. Cell suspensions were stained with a live/dead staining kit (Molecular Probes, Invitrogen) in darkness for 1 h. Fluorescence images were obtained with a Zeiss Axiovert 200M fluorescent microscope (Zeiss, German). To detect .OH using fluorescence technique, the IO NWs filter was collected and transferred into 20 mL of DI water after being operated at 4.5 V for certain time. Hydroxyl radicals were detected using a fluorescent method as previously reported (D. Wang et al., 2018). Specifically, after the bacterial cells were separated from the filter by centrifugation, the water sample was mixed with coumarin solution (10⁻³ M) for fluorescence analysis (QuantaMaster 400, PTI). To investigate the effect of .OH on the inactivation performance, dimethyl sulfoxide (DMSO) was used as a quenching agent of .OH. Specifically, the PBS solution of DMSO (1 mM, 10 mM, and 100 mM) was mixed with the suspension of S. epidermidis, respectively, which was then subject to the atomizing step. The concentration of live S. epidermidis on the IO NWs filter was then measured as described in the section of Inactivation of Bacteria. A hemocytometer was used to prove the possible lysis of S. epidermidis after treatment (condition: 4.5 V and 30 min). After treatment, the filter was resuspended in PBS buffer, which was centrifuged and concentrated into 1 mL of PBS. The produced cells pellets were stained with 0.4% crystal violet for 5 min at room temperature. After being washed with PBS buffer for three times, the pellets were resuspended into 1 mL of PBS and counted by the hemocytometer. Same protocol was employed for a controlled experiment in which no external voltage was applied (denoted as before treatment samples).

Simulation: The temperature gradient around the IO NWs filters was simulated using COMSOL Multiphysics®. The model was integrated by three parts (Equation 1-3).

∇(σ×∇V)=0 (Equation 1)

ρ×u∇u−μΔu+∇p=0 (Equation 2)

k∇²T+Cₚ×ρ×u×∇T=σ×|∇V²| (Equation 3)

The module simulated an opening of iron mesh (200 μm×200 μm), where the iron wire has a diameter of 200 μm and is covered with a layer of iron oxide (thickness=0.5 μm). The size of the air flow channel is 600 μm×600 μm×1400 μm. The meshes were made up of 39827 meshes.

Equation (1) is the solution for the electrical potential distribution in the cell, where V is the voltage and σ is the electrical conductivity of the media. Equation (2) is the classical incompressible Navier-Stokes equation, where ρ is density of air, u is velocity of air and p is the pressure. Equation (3) is the conductive and convective heat transfer equation with Joule heat as source, where k is the conductive heat transfer coefficient. T is the temperature. Cₚ is the heat capacity of air and σ×|∇V²| is Joule heating term.

The electrochemical field near the IO NWs was also simulated using the COMSOL Multiphysics® software package. The static electricity model was selected. A cubic zone with size of 26 μm×26 μm×26 μm, and a nanowire with size of 0.06 μm (radius)×13 μm (length) was simulated. The material of the cubic zone is air and the material of the nanowire is Fe₂O₃. The voltage applied on the nanowire was 4.5 V.

### Example 2: Results and Discussion

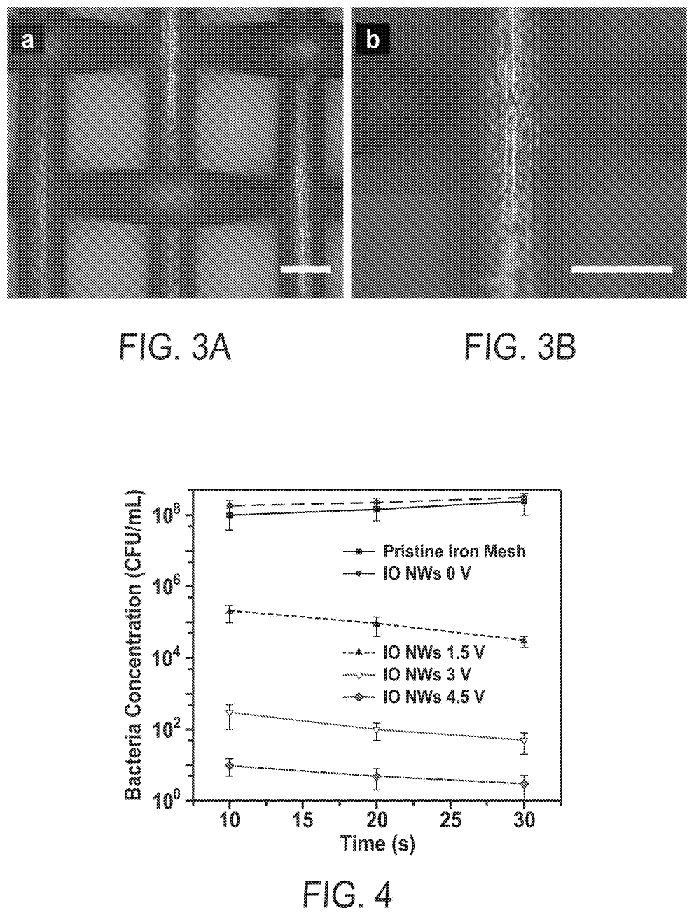

Characterization of IO NWs. Iron mesh was chosen as the substrate for the IO NWs growth because of its strong mechanical strength and potential use as the frame and/or pre-filter of conventional air filters. The pristine iron mesh (size=15×20 cm²) is of a metallic color (FIG. 2A). After thermal treatment in air at 700° C. for 6 h, the color turns burgundy in color (FIG. 2B). The optical microscopy images show that the surface of the pristine iron mesh is shiny and clean (FIGS. 3A and 3B). After thermal treatment, the surface of iron mesh is fully covered by nanowires (FIG. 2C). Closer observation reveals that the average length of the nanowires is 13 μm (FIG. 2D) and the average diameter is 120 nm (FIG. 2E). As shown in the optical microscopy image and SEM images, the coverage of the nanowires on the iron mesh is uniform and complete. To verify the composition of the nanowires, XRD analysis was carried out. As shown in FIG. 2F, the XRD patterns of the pristine iron mesh possess two peaks (20=45 and 65°), which are indexed to be metallic Fe (PDF no. 87-722).

The peaks of the sample after thermal treatment are indexed to Fe₂O₃(PDF no. 84-310), indicating that the nanowires are Fe₂O₃. Without being wed to theory, the formation of NWs at elevated temperature can potentially be attributed to the relaxation of the compressive stresses resulting from the transformation at the interface among the iron with different valence.

Inactivation Efficiency. The concentration of S. epidermidis stock suspension was ˜10⁹ CFU/mL, as determined by the standard spread plating technique. The R.H. in the chamber was maintained at 50±3%. The pristine iron mesh was considered to have little inactivation ability when the external voltage was 0 V. Under this condition, the amount of live S. epidermidis on pristine iron mesh increased slightly with a longer operation time. A similar phenomenon was also observed for the IO NWs filter (FIG. 4). A higher concentration of live S. epidermidis was also found on the IO NWs than on the pristine iron mesh, which may be due to the brush-like structure of IO NWs on the mesh opening and the increased surface area of IO NWs compared with the pristine iron wires. Due to the large opening size of the iron mesh in this study, some bacteria can escape from the IO NWs filter. The ratio of captured bacteria by IO NWs was calculated under different conditions as follows in Equation 4,

r_{captured}=N_{captured}/(N_{captured}+N_{escaped}) (Equation 4)

Where r_{captured} is the ratio of captured bacteria N_{captured} is the number of captured bacteria by the filter and N_{escaped} is the that of escaped bacteria from the filter. It was found that the capture efficiency of IO NWs filter was ˜52% at 0 V and only varied slightly with the treatment time (10-30 s), as shown in Table 1. It was also noted that the number of escaped bacteria from the filter is only dependent on treatment time, and independent on the external voltage (Table 2). Since the total amount of bacteria in the feeding air is constant for certain treatment time, the captured bacteria were also considered to be only dependent on treatment time. As a result, the log inactivation efficiency can be calculated as follows using Equation 5,

E=log(C_{(t,V)}/C_{(t,0)}) (Equation 5)

where E is the log inactivation efficiency, C_{(t,V)} is the concentration of live S. epidermidis on the IO NWs filters after the treatment at V volt and t seconds, C_{(t,0)} is the live concentration of S. epidermidis on the IO NWs filters after the treatment at 0 volt and t seconds.

TABLE 1
Calculation of capture efficiency of the two types of filters
(voltage = 0 V).

| | Treatment | N_{captured} | N_{escaped} | r_{captured} |
|---|---|---|---|---|
| Filter | time (s) | (10⁹ CFU) | (10⁹ CFU) | (%) |
| IO NWs | 10 | 3.6 | 6.8 | 52.9 |
| | 20 | 4.6 | 8.9 | 51.7 |
| | 30 | 6.4 | 12.3 | 52.0 |
| Pristine Iron | 10 | 1.3 | 7.1 | 32.4 |
| mesh | 20 | 3.1 | 10.2 | 30.4 |
| | 30 | 5.0 | 13.7 | 36.5 |

TABLE 2
The number of bacteria escaped from the IO NWs filter

| Voltage | Treatment | N_{escaped} | Percent deviation |
|---|---|---|---|
| (V) | time (s) | (10⁹ CFU) | (%, compared to 0 V) |
| 1.5 | 10 | 7.1 | 4.4 |
| | 20 | 8.7 | −2.2 |
| | 30 | 12.7 | 3.3 |
| 3 | 10 | 6.9 | 1.5 |
| | 20 | 9.3 | 4.5 |
| | 30 | 11.3 | −8.1 |
| 4.5 | 10 | 6.2 | −8.8 |
| | 20 | 9.6 | 6.7 |
| | 30 | 12.8 | 4.1 |

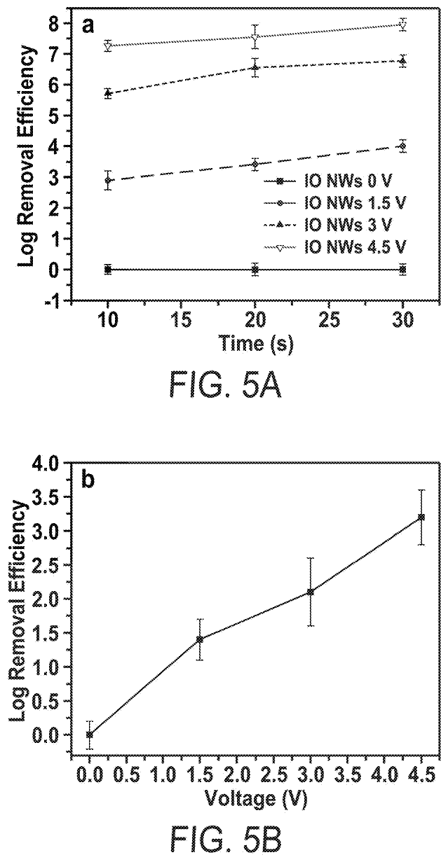

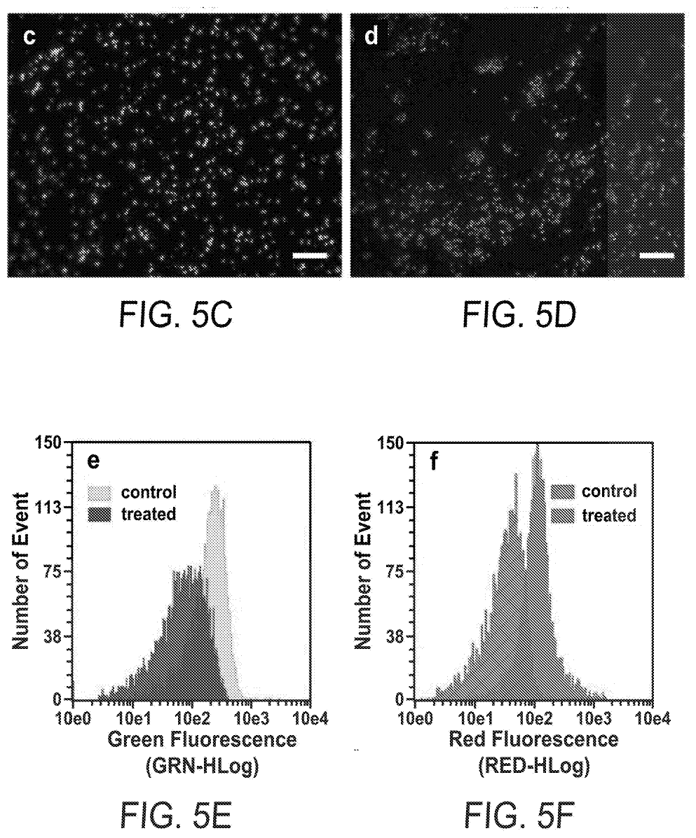

The IO NWs filters achieved ˜3 log inactivation efficiency under the condition of 1.5 V and 10 s. Notably, either increase of treatment time or applied voltage boosted the log inactivation efficiency (FIG. 5A). For example, by prolonging the treatment time from 10 s to 30 s, the log inactivation efficiency increased to ˜4. Meanwhile, by increasing the voltage from 1.5 V to 4.5 V, the log inactivation efficiency increased to >7. Considering the practical application as air filters, where a rapid inactivation performance is more desirable, the operation parameters were set to 4.5 V and 10 s for further studies. On the contrary, the pristine iron mesh filter exhibited poor capacity of inactivation compared to the IO NWs filter. Specifically, even when 4.5 V was applied, the log inactivation efficiency of the pristine iron mesh filter was ˜3.1 (FIG. 5B). The different performances between the two types of filters are discussed in the Inactivation Mechanism section.

To further confirm the inactivation performance of the IO NWs filter, the Baclight™ kit fluorescent microscopic method was employed. The live bacterial cells only accumulate SYTO 9 to emit green fluorescence, on the other hand, the dead bacterial cells accumulate both SYTO 9 and propidium iodide and emit red fluorescence. Before treatment, most of the bacterial cells exhibit green fluorescence (FIG. 5C). On the contrary, after treatment at 4.5 V for 10 s, most of the bacterial cells showed red fluorescence, indicating that the cell membrane of most S. epidermidis was damaged after treatment (FIG. 5D). The dead/live bacterial cells were analyzed using a flow cytometry. Flow cytometry records measurements from individual cells and can process thousands of cells (5,000 cells in this experiment). The area plotted in FIG. 5E and FIG. 5F represent bacterial populations that emit green and red fluorescence, respectively. As shown in FIG. 5E, flow cytometry data illustrate a left-shift of peak position, indicating that the population of live cells decreased after treatment. Similarly, the right-shift of peak position in FIG. 5F shows the population of dead cells increased after treatment.

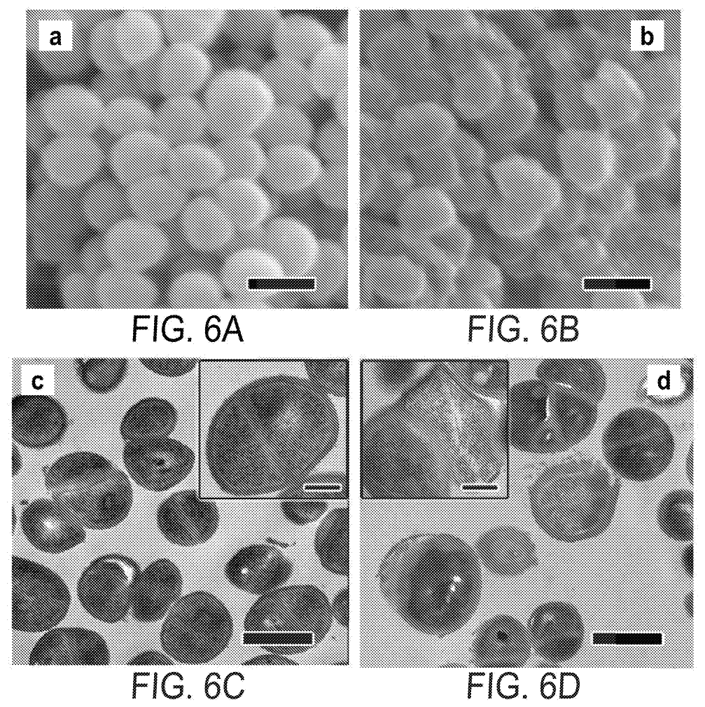

Characterization of S. epidermidis During Inactivation. The staining experiments in FIGS. 5C, 5D, 5E, and 5F indicate that the membrane integrity of treated S. epidermidis is damaged after the inactivation process. To further assess the changes of bacterial cells before and after treatment. SEM and TEM analyses were conducted. The SEM image of S. epidermidis cells before inactivation shows that the cells are of spherical shape and uniform size (FIG. 6A). Meanwhile, the surface of the bacterial cells is smooth and the membrane is complete. However, after treatment, the cellular structure of S. epidermidis experienced serious damage. Some of the cells were deformed, with shrinking of cell and leakage of cell inclusions. Some pores were also observed. Meanwhile, some other cells were broken down into debris (FIG. 6B). These changes were further confirmed by TEM analysis. As shown in FIG. 6C, S. epidermidis cells before treatment had uniform and complete cell wall structures. Meanwhile, the cytoplasm inside the cell wall was dense and homogeneous (inset in FIG. 6C). On the contrary, after treatment, many of S. epidermidis cells were seriously damaged into irregular contours (FIG. 6D). Specifically, the cell wall of some bacteria was much thinner or even seriously distorted. Some pores on the cell wall were again observed. The distorted cell wall also resulted in the less dense cytoplasm inside (inset in FIG. 6D). These electron microscope results are consistent with the results shown in FIG. 5, since only dead cells can accumulate propidium iodide and emit red fluorescence due to their disrupted cell wall.

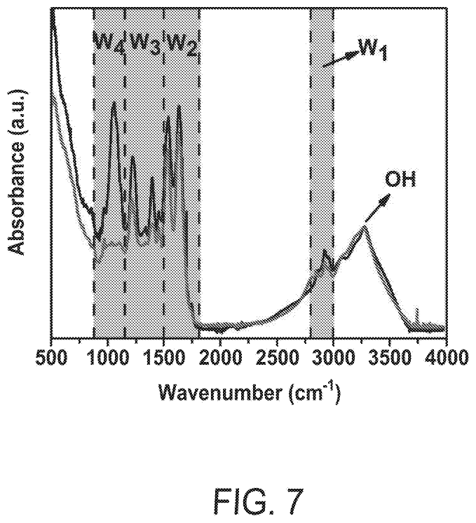

FTIR analysis of the bacteria before and after treatment was conducted because FTIR spectra comprise the vibrational characteristics of all cell constituents, including DNA/RNA, protein, membrane and cell-wall components. As shown in FIG. 7, the spectra of fresh and treated bacteria showed similar patterns. For example, the wide peaks which distribute across 3000 to 3500 cm⁻¹ correspond to the vibration of —OH due to enhanced hydration of bacteria. However, a slight change was observed in W₁ region in FIG. 7 for the bacteria after treatment. This change indicates possible damage of bacteria membrane, since W₁ is dominated by the stretching vibrations of some carbon-hydrogen bonds, which usually present in the fatty acid components of the various membrane amphiphiles. In W₂ region, even though the two major peaks remain consistent, two peak shoulders at longer wave number disappeared after inactivation process, implying the damage of proteins and peptides. It is also noted that the peak at 1335 cm⁻¹ in region W₃ weakens for bacteria after treatment. This phenomenon indicates the possible change of proteins, fatty acids and phosphate-carrying compounds. Notably, the peak at 1057 cm⁻¹ in region W₄ completely disappeared after treatment, indicating the serious damage of the carbohydrates present within the cell wall. The FTIR results were consistent with the SEM and TEM analyses.

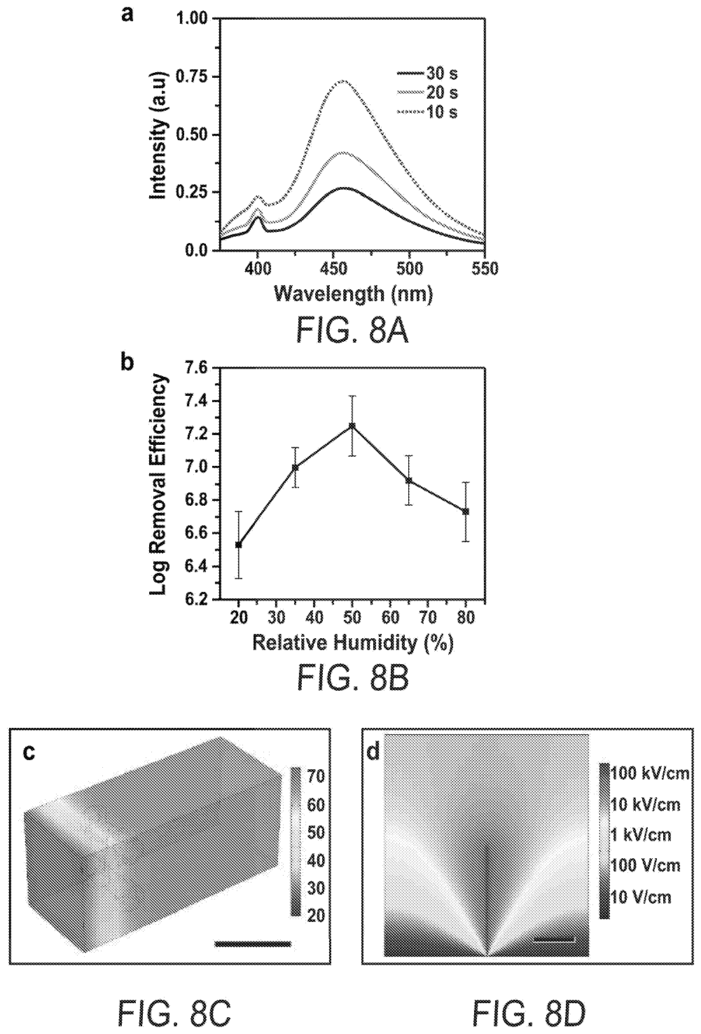

Inactivation Mechanism. Notably, .OH was found to be generated in the system. As shown in FIG. 8A, a major fluorescence peak was identified at 455 nm, which verifies the generation of .OH. The evolution of the spectra obtained at different times clearly verified the accumulation of .OH on the IO NWs filter. The production of .OH was possibly due to Fenton-like reactions since iron oxide nanomaterials can serve as strong catalysts for these reactions. As the primary agent for Fenton-like reaction, H₂O₂ can be produced through a two-electron oxygen activation, where the electrons transfer from iron core to the iron oxide shell surface. Meanwhile, it has also been reported that some electrochemical reactions among electrons, oxygen, and water are able to produce H₂O₂. The produced H₂O₂ then decomposes to generate .OH, with iron oxide as catalysts. This possible mechanism for .OH generation was further supported when no fluorescence peak was observed for the system without applying external voltage. .OH has been proven to be highly efficient to damage cells. On the other hand. H₂O₂ is a strong oxidant itself which can kill bacteria. Since iron species are important for the Fenton-like reactions, the different performance between pristine iron mesh and IO NWs mesh can be at least partially attributed to the increased surface area of IO NWs compared to pristine iron mesh. The increased surface area of IO NWs is accompanied with more exposed iron atoms, which thus facilitate the Fenton-like reactions.

Humidity is an important parameter for indoor air quality control. As such, the effect of R.H. on the inactivation performance of the filter was investigated over a range of from 20% to 80% (slightly wider than the comfortable range for human of 25-60%). The results of S. epidermidis inactivation indicated that a log inactivation efficiency of ˜6.5 was achieved at 20% R.H. (FIG. 8B). Higher inactivation efficiency was recorded when R.H. was increased to 50%. However, further increase of R.H. has a negative effect on the inactivation performance. The reduced inactivation performance of IO NWs filter at low R.H. is attributed to the low amount of water molecules available under this condition. Since water is the primary reactive agent in this system, its inadequacy can limit the production of both H₂O₂ and .OH, thus resulting into a lower inactivation performance of the system. On the contrary, when R.H. is high, multiple layers of adsorbed water can be formed on the surface of IO NWs, which reduces the number of available sites for oxygen molecules on the surface of IO NWs, thus limiting the generation of H₂O₂ and .OH.

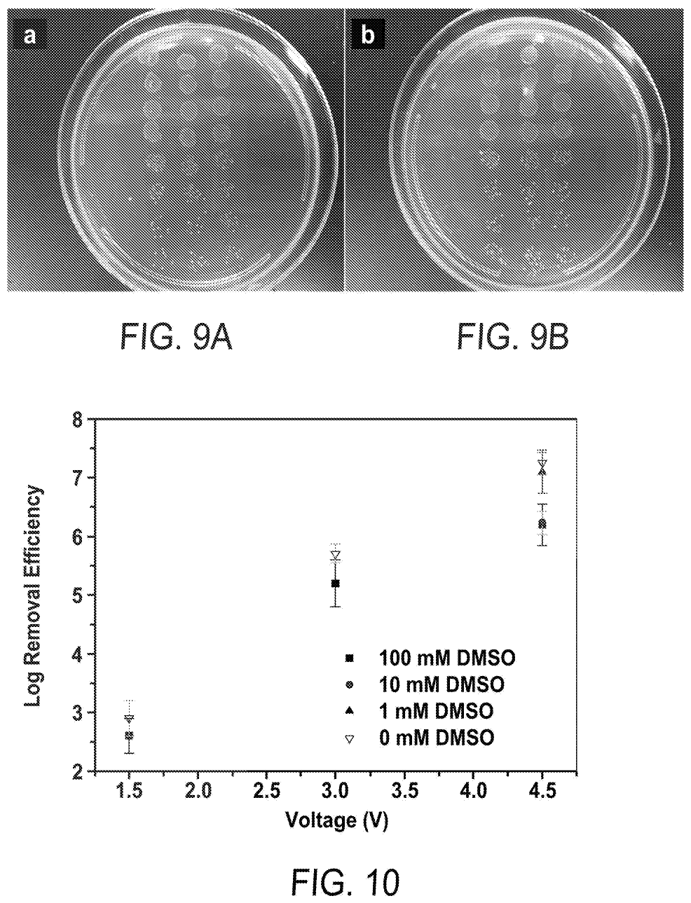

According to several previous studies employing .OH to inactivate bacteria, it usually takes tens of minutes or even hours to achieve log inactivation efficiency of >7 (Hu et al., 2010; W. Wang, et al., 2017; Li et al., 2016). Nevertheless, it only took tens of seconds to achieve such a high inactivation efficiency in the present study. This large difference suggests that other mechanisms may also be responsible for the rapid inactivation rate in this system, such as electricity and the associated Joule heating. The effects of electricity and Joule heating were elucidated by a control experiment, in which DMSO was used as the quenching agent for .OH because DMSO is non-lethal to S. epidermidis (FIG. 9A shows fresh bacteria, FIG. 9B shows bacteria treated with DMSO with no visible decrease in bacterial growth). As shown in FIG. 10, when the voltage was maintained at 4.5 V, the log inactivation efficiency decreased from 7.2 to 6.2 when the concentration of DMSO increased from 0 to 100 mM. Compared to the results shown in FIG. 5B, the presence of DMSO had limited effect on the inactivation performance. These results show that .OH produced by Fenton-like reactions only contributed in part to the rapid inactivation performance of the IO NWs filter.

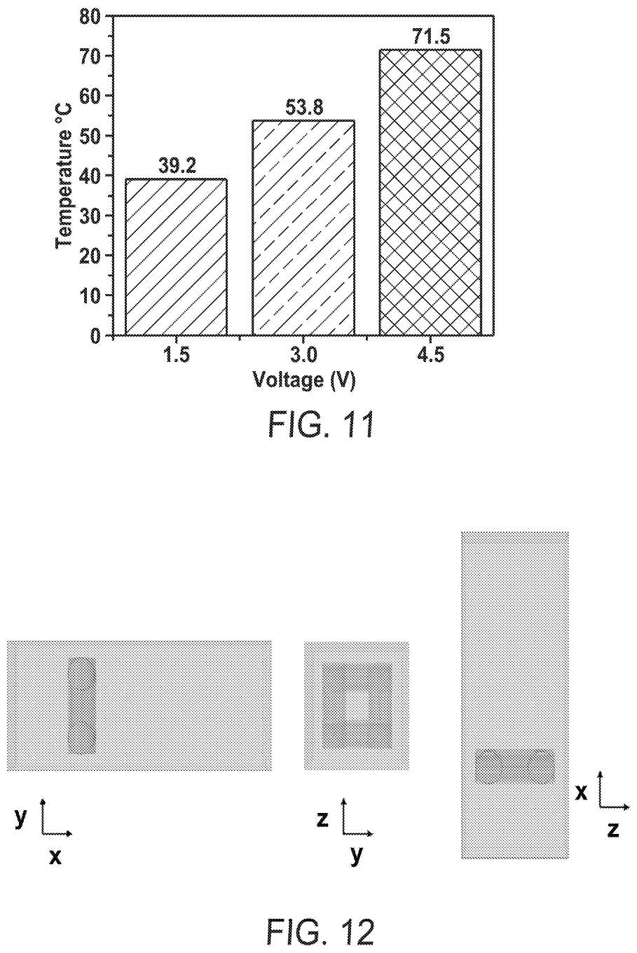

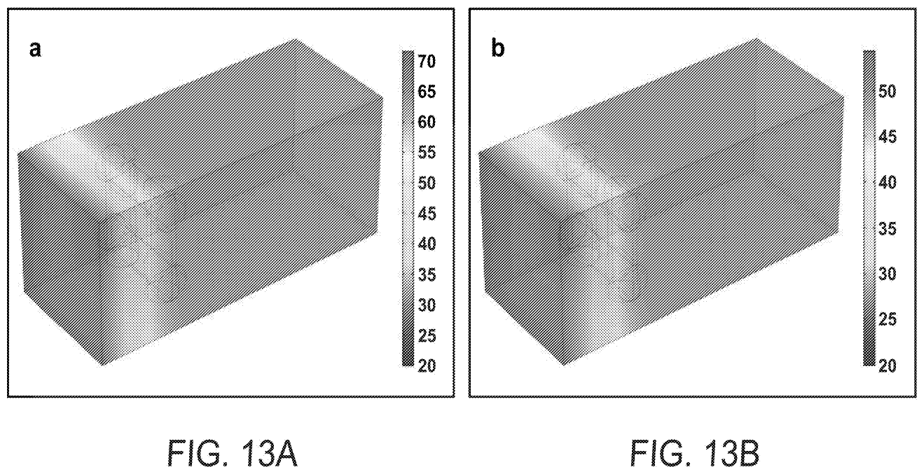

The temperature of the IO NWs filter was increased when certain voltage was applied due to the Joule heating effect. As shown in FIG. 11, the temperature of the IO NWs filter (without air flow) increased with increasing voltage. At 0 V, the temperature of the filter is close to room temperature (23.2° C.). However, the temperature increased to 71.5° C. at 4.5 V. The temperature gradient around the IO NWs filter was also calculated, showing that, not only IO NWs filter, the air in both the inflow and outflow directions were also heated (FIG. 8C). FIG. 12 shows the base structure of the iron mesh unit to be used for simulation. Simulation results are shown in FIG. 13, where the temperature distributions around the mesh structure under two different air flow rates are simulated. According to the simulation results, the temperature was increased significantly even at high air flow rate (71° C. for air velocity=0.5 m/s as shown in FIG. 13A, 59° C. for air velocity=5 m/s as shown in FIG. 13B). Thermal treatment is one of the most widely used methods for inactivation of bacteria. To elucidate the effect of Joule heating on the performance of the filter, a control experiments was conducted to exclude the effects of electricity and Fenton-like reactions. 50 μL of bacterial suspension was injected into a PCR tube (three duplicates) and then subject to thermal treatment in a thermalcycler. The samples in the tubes were heated at 71° C. for 10 s, quickly cooled down to 4° C., and then treated by standard plate culture technique. A log inactivation efficiency of >7 was measured, indicating that the effect of Joule heating on the inactivation is significant in this system.

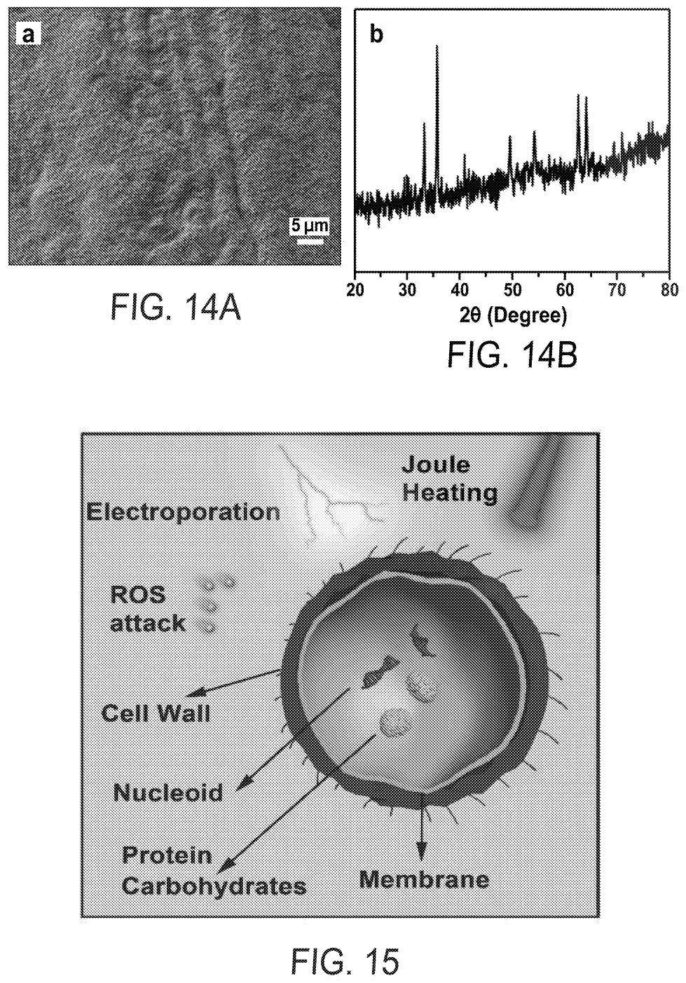

The electrical field near the IO NWs was also enhanced significantly to a magnitude of 100 kV/cm (FIG. 8D), which builds intense dipole-dipole interactions with the lipid bilayer of the cell membrane, resulting in thinning of the membrane and the introduction of electroporation pores. These phenomena were consistent with the SEM and TEM results (FIG. 3). The electroporation effect due to the NW structure was further verified by comparing the performance of IO NWs filter and IO nanoparticles (NPs) filter (FIGS. 14A and 14B). Under the same condition, the log inactivation efficiency was ˜6.4 for IO NPs filter, lower than 7.2 for IO NWs filter, suggesting that the electroporation effect resulted from NW also contributed in part to the performance of the system. Nanoparticles are spherical or somewhat spherical particles having a diameter in the nanostructure range. The diameter of nanowires may be of a similar dimension to nanoparticles, but they are much longer. The high aspect ratio of nanowires will increase the active surface area of the filter mesh. The length of the nanowire will also enhance the electric field distribution, or create a large electric field, as compared to nanoparticles, because (without being wed to theory) the electric voltage difference from tip to bottom of a nanowire is very large. The electric voltage difference is negligible for nanoparticles, as these particles have a relatively uniform size in all three dimensions. The electroporation effect also accounted for the poor performance of pristine iron mesh since it is reasonable to believe the bulk iron cannot improve the electrical field significantly.

Based on above reasons, and without being wed to theory, possible bacteria inactivation mechanisms are listed as follows. Some S. epidermidis cells can be captured by the IO NWs filter when the bioaerosols pass through the filter. In the presence of electricity, .OH was generated due to Fenton-like reactions. Meanwhile, the electrical field near the tips of IO NWs is enhanced significantly and leads to the electroporation damage of cells. The increased temperature due to Joule effect also contributed significantly to the system. All these effects worked collaboratively to damage the cell wall and nucleoid of S. epidermidis (FIG. 15) rapidly, leading to immediate death of the bacterial cells.

To further demonstrate the inactivation performance of the IO NWs filter on Gram-negative bacteria, E. coli was used as the target bacterium. A log inactivation efficiency of ˜7.6 was achieved under the operational conditions (4.5 V and 10 s, see FIG. 5A and FIG. 8B), suggesting a promising feasibility of the filter for practical applications in inactivation of both Gram-positive and Gram-negative bacteria.

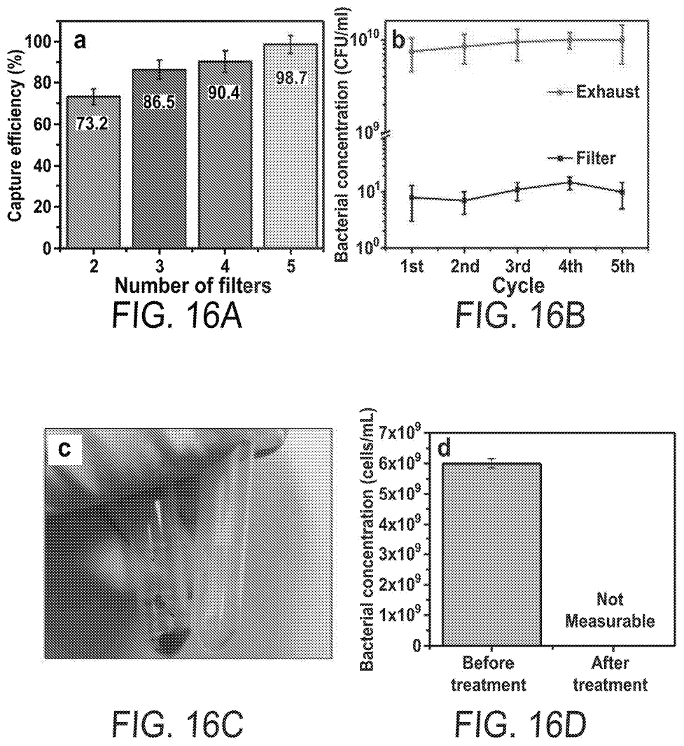

As shown above, the capture efficiency of a single IO NWs filter was ˜52%, which is low for practical applications. A higher capture efficiency can be achieved by using denser iron meshes or connecting several IO NWs filter in-tandem. The capture efficiency of the IO NWs filter was improved through the latter method. Five tandem IO NWs filters can capture 98.7% of bacteria in the air (FIG. 16A) under the experimental conditions of 4.5 V and 10 s. The performance of long-term use was also evaluated by continuously operating the system for 5 cycles (1 h for each cycle) with an external voltage of 4.5 V. The stock solution was replaced for fresh ones after each cycle, so that bioaerosol concentration was constant throughout the experiment.

After each cycle, the bacterial concentration in the exhaust buffer was counted to tell the changes of capture efficiency of the IO NWs filter. As shown in FIG. 16B, the bacterial concentration in the exhaust PBS buffer only increased slightly after each cycle, indicating that the capture capability of IO NWs filter only decreased slightly over time. This phenomenon was contrary to the expectation that the filter may be stuffed by dead bacteria so that it cannot capture any fresh bacteria. The reasonably stable capture efficiency can be ascribed to the lysis of the bacteria under the experimental conditions. Without external voltage, IO NWs filter captured a significant amount of S. epidermidis which gave an obvious pellet after being stained by crystal violet (FIG. 16C). In contrast, when 4.5 V was applied, no pellet was observed. This significant difference was also verified by counting cells by using a hemocytometer (10⁹ for 0 V and not measurable (<10⁶) for 4.5 V, FIG. 16D). Since only cells with complete cellular structure can be stained by crystal violet, these results implied that many cells may undergo lysis and occupy no space. Meanwhile, the proliferation of bacteria was not observed on the IO NWs filter over 5 cycles (FIG. 16B), showing its advantage over conventional air filter.

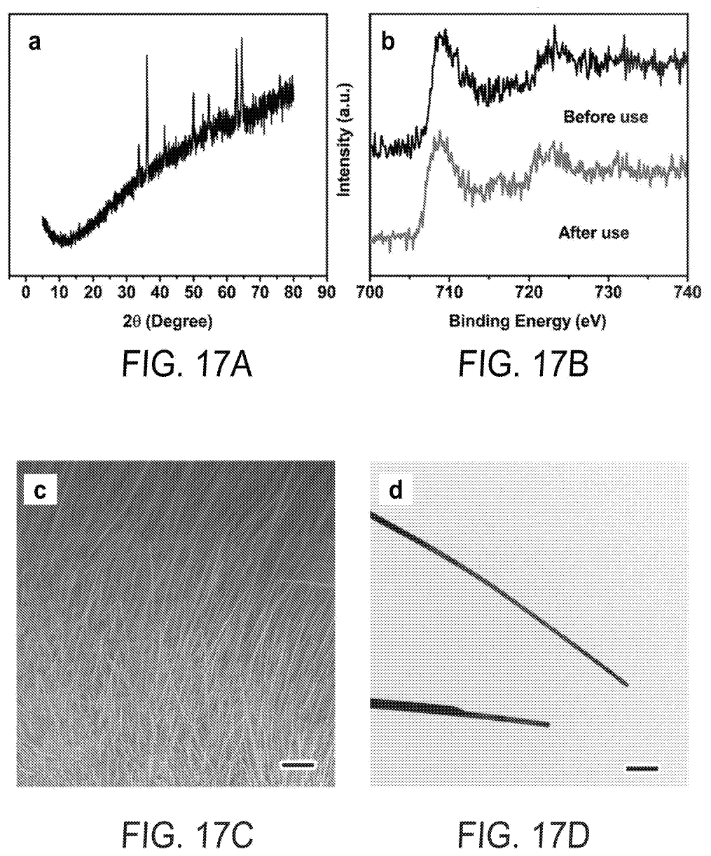

XRD, XPS, SEM, and TEM analyses of the used IO NWs filter were also conducted for the filter after five cycles of 1 h operation (FIG. 17). As shown in FIG. 17A, the peaks indexed to Fe₂O₃ were clearly identified. Meanwhile, XPS spectra of the filter before and after 1 h operation were also found to be similar (FIG. 17B). The SEM (FIG. 17C) and TEM images (FIG. 17D) also verified that the nanowire morphology was maintained after recycle use. The above results demonstrated that the IO NWs filter had a satisfactory structural stability under the experimental conditions.

In summary, an IO NWs-based filter has been developed for the control of indoor bioaerosols. A log inactivation efficiency of >7 was achieved towards S. epidermidis within 10 s when the filter was applied with a voltage of 4.5 V. The .OH, the electroporation effect, and the Joule heating were accounted for the rapid inactivation of S. epidermidis. The filter also demonstrated promise of improved capture capability and satisfactory long-term performance. The robust synthesis and satisfactory inactivation performance of the filter make it promising for HVAC filtration systems as an antibacterial layer (e.g. assembled into conventional airfilters).

### REFERENCES

1. N. E. Klepeis, W. C. Nelson, W. R. Ott, J. P. Robinson, A. M. Tsang, P. Switzer, J. V. Behar, S. C. Hern and W. H. Engelmann, J Expo Anal Environ Epidemiol, 2001, 11, 231-252.
2. D. Dai, A. J. Prussin, L. C. Marr, P. J. Vikesland, M. A. Edwards and A. Pruden, Environ. Sci. Technol., 2017, 51, 7759-7774.
3. A. P. Jones, Atmos. Environ., 1999, 33, 4535-4564.
4. Z. D. Bolashikov and A. K. Melikov, Build Environ., 2009, 44, 1378-1385.
5. P. Azimi, D. Zhao and B. Stephens, Atmos. Environ., 2014, 98, 337-346.
6. M. Möritz H. Peters. B. Nipko and H. Rüden, Int.l J. Hyg. And Environ. Health, 2001, 203, 401-409.
7. J. B. Harstad, H. M. Decker and A. G. Wedum, Appl. Microbiol., 1954, 2, 148-151.
8. N. G. Reed, Public Health Reports, 2010, 125, 15-27.
9. X. Hu, C. Hu. T. Peng, X. Zhou and J. Qu, Environ. Sci. Technol., 2010, 44, 7058-7062.
10. H. Shi, G. Li. H. Sun, T. An, H. Zhao and P.-K. Wong, Appl. Catal., B, 2014, 158, 301-307.
11. A. Vohra, D. Y. Goswami, D. A. Deshpande and S. S. Block, Appl Catal., B, 2006, 64, 57-65.
12. Y. Liang, Y. Wu, K. Sun, Q. Chen, F. Shen, J. Zhang, M. Yao, T. Zhu and J. Fang, Environ. Sci. Technol., 2012, 46, 3360-3368.
13. Y. Wu, Y. Liang, K. Wei, W. Li, M. Yao, J. Zhang and S. A. Grinshpun, Appl. Environ. Microbiol., 2015, 81, 996-1002.
14. H. Zhang, L. Yang, Z. Yu and Q. Huang, J. Hazard. Mater., 2014, 268, 33-42.
15. Q. Zhang, B. Damit, J. Welch, H. Park, C. Y. Wu and W. Sigmund, J Aerosol Sci, 2010, 41, 880-888.
16. M. H. Woo, A. Grippin, C. Y. Wu and J. Wander, Aerosol Air Qual. Res., 2012, 12, 295-303.
17. C. E. Yale and A. R. Vivek, Appl. Microbiol., 1968, 16, 1650-1654.
18. E. Miaskiewicz-Peska and M. Lebkowska, Fibres Text. East. Eur, 2011, 19, 73-77.
19. B. Del Curto, P. Tarsini and A. Cigada, Journal of Applied Biomaterials & Functional Materials, 2016, 14, 0-0.
20. Y. H. Joe, K. Woo and J. Hwang, J. Hazard. Mater., 2014, 280, 356-363.
21. Y.-S. Ko, Y. H. Joe, M. Seo, K. Lim, J. Hwang and K. Woo, J. Mater. Chem. B, 2014, 2, 6714-6722.
22. C. W. Park and J. Hwang, J. Hazard. Mater., 2013, 244, 421-428.
23. Z.-Y. Huo, X. Xie, T. Yu, Y. Lu, C. Feng and H.-Y. Hu, Environ. Sci. Technol., 2016, 50, 7641-7649.
24. W. Wang, T. W. Ng, W. K. Ho, J. Huang, S. Liang, T. An, G. Li, J. C. Yu and P. K. Wong, Appl. Catal., B, 2013, 129, 482-490.
25. Y. Fu, J. Chen and H. Zhang, Chemical Physics Letters, 2001, 350, 491-494.
26. L. Yuan, Y. Wang, R. Cai, Q. Jiang, J. Wang, B. Li, A. Sharma and G. Zhou, Materials Science and Engineering: B, 2012, 177, 327-336.
27. Y. Fu, J. Chen and H. Zhang, Chem. Phys. Lett. 2001, 350, 491-494.
28. Q. Zhang, R. Ma, Y. Tian, B. Su, K. Wang, S. Yu, J. Zhang and J. Fang, Environ. Sci. Technol., 2016, 50, 3184-3192.
29. A. M. A-Hashimi, T. J. Mason and E. M. Joyce, Environ. Sci. Technol., 2015, 49, 11697-11702.
30. X. Nie, W. Liu, M. Chen, M. Liu and L. Ao, Front. Environ. Sci. Eng., 2016, 10, 12.
31. D. Naumann, D. Helm and H. Labischinski, Nature, 1991, 351, 81-82.
32. R. K. Singh, L. Philip and S. Ramanujam, RSC Adv., 2016, 6, 11980-11990.
33. D. Helm, H. Labischinski, G. Schallehn and D. Naumann, Microbiology, 1991, 137, 69-79.
34. K.-i. Ishibashi, A. Fujishima, T. Watanabe and K. Hashimoto, Electrochem. Commun., 2000, 2, 207-210.
35. C. Cai, Z. Zhang, J. Liu, N. Shan, H. Zhang and D. D. Dionysiou, Appl Catal., B 2016, 182, 456-468.
36. Y. Liu, X. Liu, Y. Zhao and D. D. Dionysiou, Appl. Catal., B, 2017, 213, 74-86.
37. J. Shi, Z. Ai and L Zhang, Water Res., 2014, 59, 145-153.
38. J. Casado, J. Fornaguera and M. I. Galán, Environ. Sci. Technol., 2005, 39, 1843-1847.
39. H. Lim, J. Lee, S. Jin, J. Kim, J. Yoon and T. Hycon. Chem. Commun., 2006. DOI: 10.1039/B513517F, 463-465.
40. L. Rizzo. A. Della Sala, A. Fiorentino and G. Li Puma, Water Res., 2014, 53, 145-152.
41. V. J. P. Vilar, C. C. Amorim, E. Brillas, G. L. Puma, S. Malato and D. D. Dionysiou, Environ Sci Pollut R, 2017, 24, 5987-5990.
42. García-Fernández, M. I. Polo-López, I. Oller and P. Fernández-Ibáñez, Appl. Catal., B, 2012, 121, 20-29.
43. A. L. Goodman, E. T. Bernard and V. H. Grassian, J. Phys. Chem. A, 2001, 105, 6443-6457.
44. Y. Li. C. Zhang, D. Shuai, S. Naraginti, D. Wang and W. Zhang, Water Res., 2016, 106, 249-258.
45. W. Wang, G. Li, D. Xia, T. An, H. Zhao and P. K. Wong, Environ. Sci. Nano. 2017, 4, 782-799.
46. Y. Hong. L. Li. G. Luan, K. Drlica and X. Zhao, Nat. Microbiol., 2017, DOI: 10.1038/s41564-017-0037-y.
47. W. A. Moats, J. Bacteriol., 1971, 105, 165-171.
48. M. Yao, G. Mainelis and H. R. An, Environ. Sci. Technol., 2005, 39, 3338-3344.
49. C. Liu, X. Xie, W. Zhao, N. Liu, P. A. Maraccini, L. M. Sassoubre, A. B. Boehm and Y. Cui, Nano Lett., 2013, 13, 4288-4293.
50. D. T. Schoen, A. P. Schoen, L. Hu, H. S. Kim, S. C. Heilshorn and Y. Cui, Nano Lett., 2010, 10, 3628-3632.
51. T. Kotnik, W. Frey, M. Sack, S. Haberl Meglič, M. Peterka and D. Miklavčič, Trends Biotechnol., 2015, 33, 480-488.

1. A filtration system comprising:
a filter mesh, the filter mesh comprising a porous lattice of iron metal and iron oxide nanowires radiating from the porous lattice of iron metal.

2. The filtration system according to claim 1, wherein the iron oxide nanowires have a diameter of no more than 300 nanometers.

3. The filtration system according to claim 1, wherein the nanowires have length of at least 3 micrometers.

4. The filtration system of claim 1, wherein the porous lattice comprises reactive oxygen species.

5. The filtration system according to claim 1, further comprising a housing having an inlet and an outlet, the filter mesh being disposed between the inlet and the outlet.

6. The filtration system according to claim 5, further comprising a plurality of filter meshes arranged in sequence between the inlet and the outlet.

7. The filtration system according to claim 5, further comprising at least three filter meshes arranged in sequence between the inlet and the outlet.

8. The filtration system according to claim 5, further comprising a power supply in electrical communication with the filter mesh and configured to apply a voltage to the filter mesh.

9. A method for the inactivation of pathogens, comprising:
providing a filter mesh comprising a porous lattice of iron metal and iron oxide nanowires radiating from the porous lattice of iron metal;
passing a sample containing pathogens through the filter mesh; and
inactivating at least a portion of the pathogens as the sample passes through the filter mesh.

10. The method of claim 9, wherein inactivating at least a portion of the pathogens comprises lysing pathogen cell membranes.

11. The method of claim 9, wherein passing the sample through the filter mesh further comprises passing the sample through a plurality of filter meshes arranged in sequence.

12. The method of claim 9, further comprising applying a voltage to the filter mesh.

13. The method of claim 12, wherein the voltage is at least 0.1 V.

14. The method of claim 9, further comprising heating the filter mesh.

15. The method of claim 9, wherein inactivating at least a portion of the pathogens further comprises inactivating Gram-positive bacteria.

16. The method of claim 9, wherein inactivating at least a portion of the pathogens further comprises inactivating Gram-negative bacteria.

17. A method of manufacturing a filter mesh, comprising:
providing a porous lattice of iron metal;
washing the porous lattice of iron metal with hydrochloric acid;
rinsing the porous lattice of iron metal with water;
drying the porous lattice of iron metal; and
heating the porous lattice of iron metal to a temperature ranging from 600° C. to 900° C.

18. The method of claim 17, wherein the hydrochloric acid is at least 0.1 M hydrochloric acid.

19. The method of claim 17, wherein the drying is performed with a vacuum desiccator.

20. The method of claim 17, wherein the porous lattice of iron metal is heated for a time period of from 5 hours to 7 hours.

21. The method of claim 17, wherein the heating occurs at a rate wherein the temperature rises by about 3° C./minute to about 10° C./minute.
